(19) United States
(12) Patent Application Publication
(10) Pub. No.: US 2021/0375454 A1
(43) Pub. Date: Dec. 2, 2021

(54) AUTOMATED OPERATORS IN HUMAN REMOTE CAREGIVING MONITORING SYSTEM

(71) Applicant: Cherry Labs, Inc., Wilmington, DE (US)

(72) Inventors: Maksim Goncharov, Redwood City, CA (US); Vasiliy Morzhakov, Moscow (RU); Stanislav Veretennikov, San Francisco, CA (US)

(21) Appl. No.: 17/403,616

(22) Filed: Aug. 16, 2021

Related U.S. Application Data
(63) Continuation-in-part of application No. PCT/US21/24334, filed on Mar. 26, 2021.
(60) Provisional application No. 63/001,869, filed on Mar. 30, 2020.

(51) Int. Cl. G16H 40/67; G16H 50/20; G06F 21/62; G06K 9/62

(52) U.S. Cl. CPC G16H 40/67; G06K 9/6218; G06F 21/6254; G16H 50/20

(57) ABSTRACT

A method includes receiving a data stream from an input device at a monitored location. The data stream is processed to determine whether an abnormal event has occurred. The method further includes transmitting data associated with whether the abnormal event has occurred to a user. Data associated with user actions in response to the transmitting data is collected. The method finally includes generating a machine learning model based on the received data stream, the processed data stream and whether the abnormal event has occurred, and further the collected data associated with user actions in response to the transmitting.

### CROSS-REFERENCE TO RELATED APPLICATIONS

This application is a continuation application of United States Patent Application No. PCT/US21/24334, filed Mar. 26, 2021, entitled “System and Method for Efficient Machine Learning Model Training,” which claims the benefit of U.S. Provisional Patent Application No. 63/001,869, filed Mar. 30, 2020. Both of which are incorporated herein in their entireties by reference.

### BACKGROUND

A variety of security, monitoring and control systems equipped with a plurality of cameras and/or sensors have been used to detect various threats such as health threats (e.g., falling, fainting, becoming unconscious and unresponsive, etc.) as well as security threats such as intrusions, or even natural disaster threats such as fire, smoke, flood, etc. For a non-limiting example, motion detection is often used to detect intruders in vacated homes or buildings, wherein the detection of an intruder may lead to an audio or silent alarm and contact of security personnel. Video monitoring is also used to provide additional information about personnel living in an assisted living facility but unfortunately it is labor intensive.

Currently, the monitoring and control systems may detect an event occurrence, and an operator is notified and alerted. The operator may then decide on the appropriate course of action, e.g., notifying 911, notifying a family member, notifying the police, notifying a healthcare professional, etc. Unfortunately, once an event has occurred the process becomes manual in nature since a human intervention is required to make a decision on the appropriate course of action.

The foregoing examples of the related art and limitations related therewith are intended to be illustrative and not exclusive. Other limitations of the related art will become apparent upon a reading of the specification and a study of the drawings.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 depicts a block diagram of a monitoring system in accordance with some embodiments.

FIG. 2 depicts an application example of a monitoring system in accordance with some embodiments.

FIG. 3 depicts an application example of a monitoring system detecting an abnormal event in accordance with some embodiments.

FIG. 4 depicts an application example of a monitoring system rendering events in accordance with some embodiments.

FIG. 5 depicts an application example of selecting a portion of the captured data to be transmitted for further analysis or for alerting an individual in accordance with some embodiments.

FIG. 6 depicts relational node diagram depicting an example of a neural network for generating a machine learning model in accordance with some embodiments.

FIG. 7 depicts a flow chart illustrating an example of method flow for generating a machine learning model in accordance with some embodiments.

FIG. 8 depicts a block diagram depicting an example of computer system suitable for generating a machine learning model and monitoring system in accordance with some embodiments.

### DETAILED DESCRIPTION OF EMBODIMENTS

The following disclosure provides many different embodiments, or examples, for implementing different features of the subject matter. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

A new approach is proposed that contemplates systems and methods to monitor premises, e.g., home, office facility, manufacturing floor, healthcare facility, nursing home, etc., to detect an abnormal event at the premises, e.g., fire, smoke, flood, intrusion, fall, stroke, etc., in a smart fashion by leveraging machine learning (ML) model. In some embodiments, the ML model may either be trained under supervision via provided training data or be trained without supervision and over time by analyzing the behaviors and patterns within the monitored premises.

In general, a monitoring system may identify various actionable events, which are also referred to as abnormal event in this application. The actionable events are generally transmitted to an operator to make a decision and take appropriate actions. For a non-limiting example, when the actionable or abnormal event is a fall or a stroke then a call to 911 or an ambulance may be initiated, whereas for fire and smoke the fire department may be notified and in a case of home invasion the police department is notified, etc. In other instances, the operator may initiate a call to a family member, or may transmit a portion of the captured video/audio data to another entity, send an email, initiate a two way communication with a person at a monitored location, send a text, etc. The decision and the actions taken by the operator are manual in nature. Under a ML-driven monitoring system, the ML model is generated based on the monitored data, e.g., audio/video stream of data, that is in some embodiments captured from a monitored location, further based on various abnormal event as processed by a processing unit, and further based on the actions taken by the operator. In other words, the ML model learns from appropriate actions taken by the operator and once applied in the field can emulate a similar response or appropriate action.

In some embodiments, the ML model is generated based on the monitored data at a different location (e.g., in a control setting or from other users) from that of the location being monitored. The ML model may be generated based on abnormal events as determined by processing the monitored data or by monitored data that is tagged as such with a list of appropriate actions. In other words, the ML model may be generated in a supervised fashion.

Once the ML model is generated it may be stored. The ML model may be applied to the processed data that determine whether an abnormal event has occurred and to identify appropriate actions to be taken. In other words, the need for an operator to manually decide on the appropriate course of action and to take that action is eliminated.

Although security monitoring systems have been used as non-limiting examples to illustrate the proposed approach to efficient ML model training, it is appreciated that the same or similar approach can also be applied to efficiently train and validate ML model used in other types of AI-driven systems.

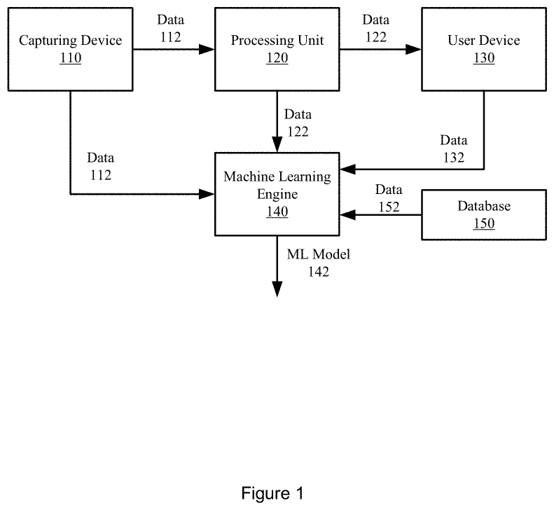

FIG. 1 depicts a block diagram of a monitoring system in accordance with some embodiments. The monitoring system may include a capturing device 110, a processing unit 120, a user device 130, a machine learning engine 140, and a database 150. In some embodiments, an input data, e.g., audio, video, etc., is captured by the capturing device 110, e.g., camera, microphone, infrared, etc. The captured data 112 may be processed or transmitted to the processing unit 120 and the machine learning engine 140 without processing. In some nonlimiting examples, for privacy reasons certain portions of the captured data 112 is pixelated or a 2-dimentional (2-D) image (e.g., skeletons) of a person in the captured video may be generated to protect the individual's privacy. The processing unit 120 processes the captured data 112 to determine whether an abnormal event has occurred, e.g., a fall, a stroke, fire, flood, smoke, home invasion, medical condition, etc. It is appreciated that in some embodiments the captured data 112 is processed to determine the individual's pose, position, orientation, height position, etc., which are critical in identifying the person's ordinary/normal activities at the monitored location. It is appreciated that in some embodiments, the captured data 112 or a modified version thereof, e.g., 2D images of a person, etc., may be stored in a storage medium, e.g., hard drive, solid state drive, etc. It is appreciated that replacing an individual in the image with a 2D image may significantly reduce the processing needs of the system, e.g., less processing resources may be needed, processing speed may be increased, etc.

In some embodiments, the processing unit 120 determines whether an abnormal event has occurred based on the processed information, e.g., individual's pose, position facial feature, orientation, audio, etc. According to some embodiments, the processing unit 120 applies a machine learning model to determine whether an abnormal event has occurred. For example, the machine learning model may be used to compare the processed data to that of prior events and if a divergence from prior events is detected (e.g., divergence from normal detected pattern) then the processing unit 120 may determine that an abnormal event has occurred. In some embodiments, the ML model may include a neural network model for clustering, grouping, etc. The processing unit 120 generates data 122 that is associated with whether an abnormal event has occurred. The generated data 122 is transmitted to the machine learning engine 140 as well as the user device 130 that is associated with an operator.

In some embodiments, the operator makes a decision on the appropriate actions and steps to be taken, e.g., notifying a family member, emailing a healthcare professional, calling 911, initiating a police dispatch, initiating a two way communication with the individual being monitored, sending a text, sending an email, etc. The appropriate actions and steps as determined by the operator and performed on the user device 130 is tracked and the data 132 associated therewith is transmitted to the machine learning engine 140.

It is appreciated that in some embodiments, the database 150 stores various events that are tagged as abnormal events (from the same location being monitored or from other locations and users). Moreover, the database 150 may store various actions associated with each of the tagged abnormal events. The data 152 stored in the database 150 may also be transmitted to the machine learning engine 140.

The machine learning engine 140 therefore receives data 112 from the capturing device 110, data 122 from the processing unit 120, data 132 from the user device 130 that is associated with actions and steps taken by the operator, and/or data 152 from the database 150. Based on the received data or a combination thereof, the machine learning engine 140 generates a ML model 142 to emulate appropriate actions to be taken based on the determined abnormal event and further based on the captured/monitored data. It is appreciated that the machine learning model 142 may be trained based on the data from other individuals from other premises and/or based on collecting data from the location where monitoring is being conducted over time. For example, the machine learning model 142 functions differently on a premises with a toddler that falling is a regular occurrence than premises without one or with seniors. Once trained, the one or more machine learning model is applied by the monitoring system to filter one or more video/audio data streams of captured daily activities at the monitored location and to determine and perform the appropriate actions. It is appreciated that the appropriate actions as determined and performed emulate what an operator would have done under those circumstances but since the machine learning model is being used, the need for the operator is eliminated.

It is appreciated that the machine learning model may be modified over time as the behavior of the individuals at the monitored premises change and further as the appropriate actions to be taken changes over time. In other words, the monitoring system tracks the short term as well as long term behavioral trends within the monitored location by monitoring changes. In some examples, the manner of which the machine learning model behaves changes as the monitored location, e.g., individuals at the monitored location, changes. For example, in some embodiments, the machine learning model may behave differently before an individual at a monitored location has a stroke and after because the facial features, the pose, the orientation, the way the body moves, the positioning of the individual, the height of the individual (e.g., if now wheelchair bound), etc.

When applied specifically to a non-limiting example of home monitoring pertinent to elderly care, the proposed approach enables all normal routine activities/events/behaviors of the elders to be quickly learned by the ML model in order to ascertain the daily normal behavior, which will be tagged accordingly. Although the daily normal activities are usually immensely complex to learn, analyze and predict, and to determine appropriate actions to act upon, the proposed approach is able to drastically reduce the time it takes to train and deploy the ML model for a neural network from a captured video stream to expeditiously determine the appropriate actions to be taken. As such, when integrated into a security monitoring system, the trained ML model can effectively and efficiently detect subtle abnormal trends in the daily activities of the elders, such as a person is walking slower, starting to limp over a period of time (e.g., 6 to 12 months), waking up more frequently during the night, etc., and to determine the appropriate actions to be taken. In some embodiments, the ML model can be quickly trained and generated to correlate certain appropriate actions (by the operator) to specific abnormal events like falling, coughing, distress, etc. As such, once deployed with real data the ML model 142 can quickly decide on the appropriate action to be taken that is specific to the monitored premises.

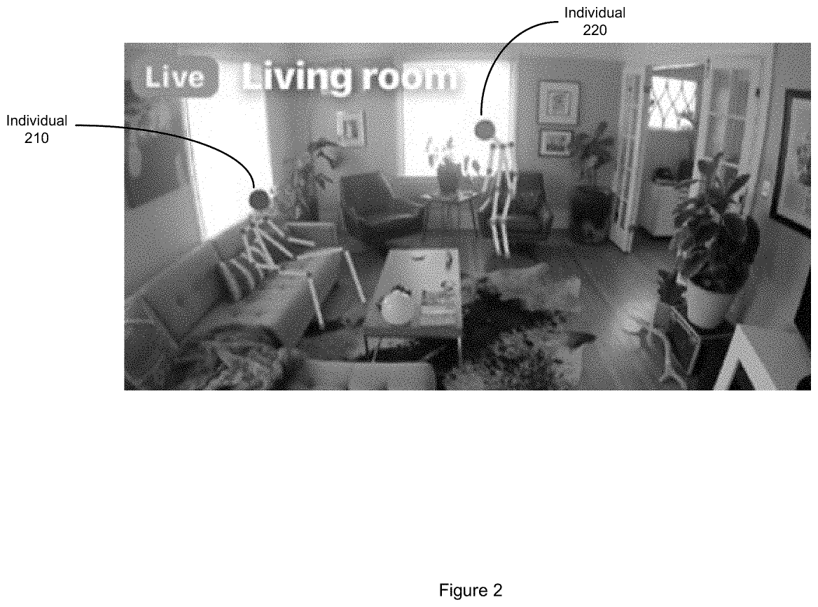

FIG. 2 depicts an application example of a monitoring system in accordance with some embodiments. In this example, the monitoring system is monitoring a monitored location, e.g., living room. In this example, two individuals are present, individuals 110 and 120. The individuals are represented as a 2-D image for illustrative purposes. According to some embodiments, the identity of the individuals is obfuscated, e.g., by rendition in 2-D images, or pixelated, etc., in order to protect their privacy, e.g., in response to a privacy signal indication a desire to be in private mode. It is appreciated that in other embodiments, the individuals may be represented in as 2-D images in order to reduce the processing complexity and the processing resources of the computing system. In this illustrative example, individual 110 is seated while individual 120 is standing.

It is appreciated that the premises may be monitored in order to determine whether an abnormal event has occurred. Moreover, it is appreciated that as more and more data, e.g., video/audio data, is collected and processed, the accuracy of the monitoring system in determining whether an abnormal event has occurred increases.

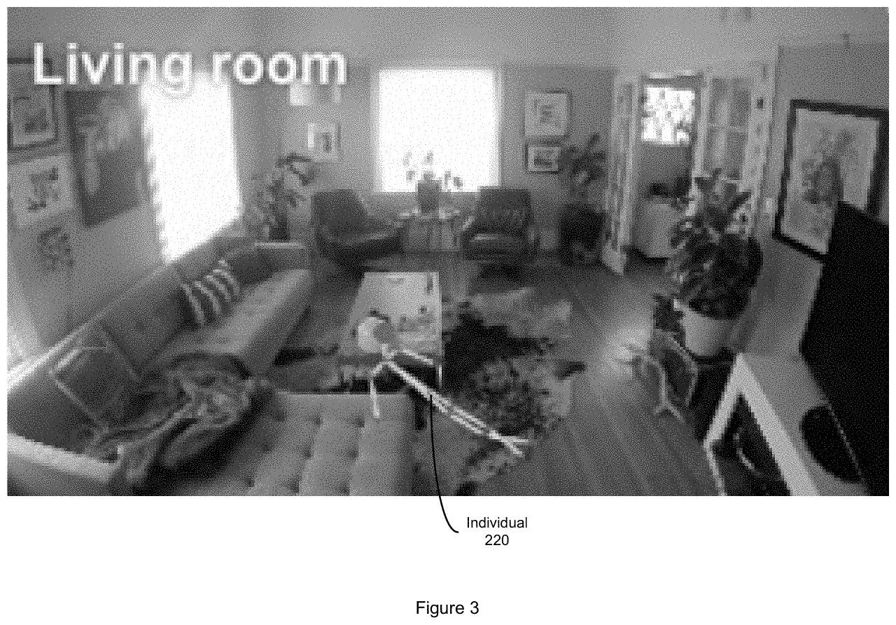

It is appreciated that monitored data (i.e. video data stream and audio data stream in this example) may be collected from the capturing device 110. In this illustrative example, the data that has been collected is provided to the ML model to determine whether an abnormal event/behavior has occurred. Referring now to FIG. 3, the monitoring data reveals that individual 120 has fallen on the floor. The data 112 is sent to the processing unit 120 that determines the event (i.e. fall) as an abnormal event.

In some embodiments, the data 122 associated with the abnormal event is transmitted to the user device 130 associated with the operator. The data 122 is also transmitted to the machine learning engine 140. The machine learning engine 140 also receives the monitoring data 112. The actions and steps taken by the operator is tracked and monitored by the user device 130 and transmitted as data 132 to the machine learning engine 140. The machine learning engine 140 uses the received data to generate a machine learning model 142 that emulates the operator. As such, once the machine learning model 142 is trained and generated and once it is deployed in the field it determines the appropriate actions to be taken for each monitored location, as if those appropriate actions were being taken by an operator. The machine learning model 142 may be a neural network and include various models for clustering, grouping, pattern recognition, etc.

It is appreciated that while in this particular example falling is identified as an abnormal event or behavior and the appropriate action to it may be calling 911 in other examples it may not. For a non-limiting example, the same scenario of an individual tripping and falling may not be as alarming when a toddler is learning to walk in comparison to when an elderly person is tripping and falling. In other words, the ML model 142 is tailored based on the individuals being monitored and as such the appropriate actions to be taken is tailored toward the specific constraints of the location being monitored. In other words, the ML model 142 does not apply a one size fit all approach but rather tailors the processing based on the specifics associated with the premises being monitored and processed.

As yet another non-limiting example, an individual with Alzheimer's that may need around the clock care may be monitored. Monitoring the premises and processing the captured data may reveal that the caretaker has left the premises and that the individual is alone. As such, based on the past behavior and knowledge by the ML model that this individual needs around the clock care, a determination is made that an abnormal event/behavior has occurred and that the appropriate action is to notify someone, e.g., caretaker, family member, etc.

It is appreciated that in some embodiments, the training data used to train the ML model may not be changed or modified over time based on the individual's behavior and/or activity within the monitored premises. As such, description of the ML model being modified over time based on the data being collected at the monitored location is for illustrative purposes and should not be construed as limiting the scope.

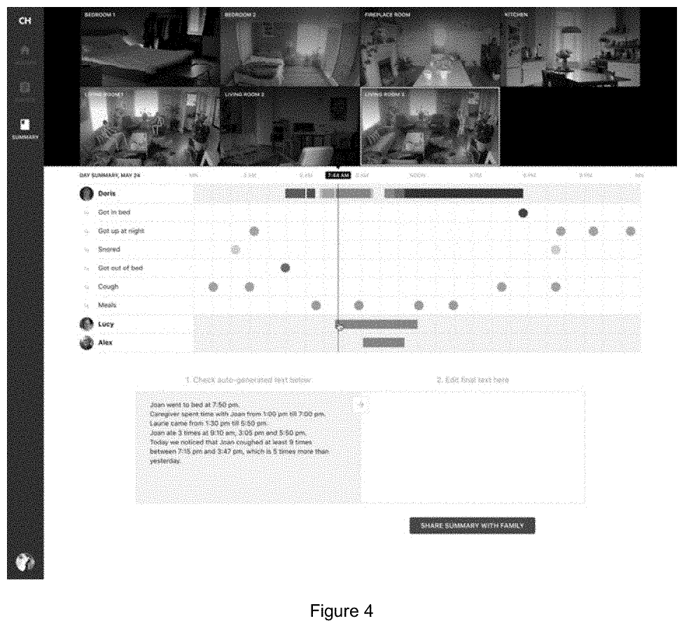

FIG. 4 depicts an application example of a monitoring system rendering events in accordance with some embodiments. The illustrated dashboard may display events throughout the day, weeks, months, etc. In this example, the monitored premises include two bedrooms, one fireplace room, one kitchen, and three living rooms. For each of the monitored location, e.g., kitchen, various events for each individual may be logged. For example, in this illustrative embodiment, Doris' activities have been monitored and logged, e.g., got in bed, got up at night, snored, got out of bed, cough, meals, etc. In some embodiments, the tracked activities may be dynamically changed by the user, operator, or family member. In some embodiments, the monitoring system may automatically modify the activities being tracked based on the individual's present and/or past behavior. For example, if an individual has never done a certain thing, e.g., stopped breathing at night while sleeping due to sleep apnea, then that occurrence may be tracked and logged. Similarly, individual's habits are also tracked, e.g., waking up in the morning on particular time during the week as opposed to weekends, etc. In other words, the monitoring may automatically determine what activity needs to be tracked and monitored and it automatically may make appropriate changes to what is to be tracked and what is to be ignored.

In this illustrative example of FIG. 4, a number of times and the particular time during the day that an event has occurred may be tracked and displayed when requested. In this particular example, Doris has got up at night 4 times, at 7:45 pm, 9:30 pm, 11:30 pm, and 3:15 am. Similarly, number of times that Doris has snored and the time may be tracked and logged. It is appreciated that various activities are tracked for each individual at the monitored premises and may be displayed on the dashboard, when requested. The logged information may be provided to the processor 120 to determine an abnormal event has occurred, as described above. The processed information may be transmitted to the user device 130, as described above, as well as the machine learning engine 140 in order for the machine learning engine 140 to generate the machine learning model 142, as described above.

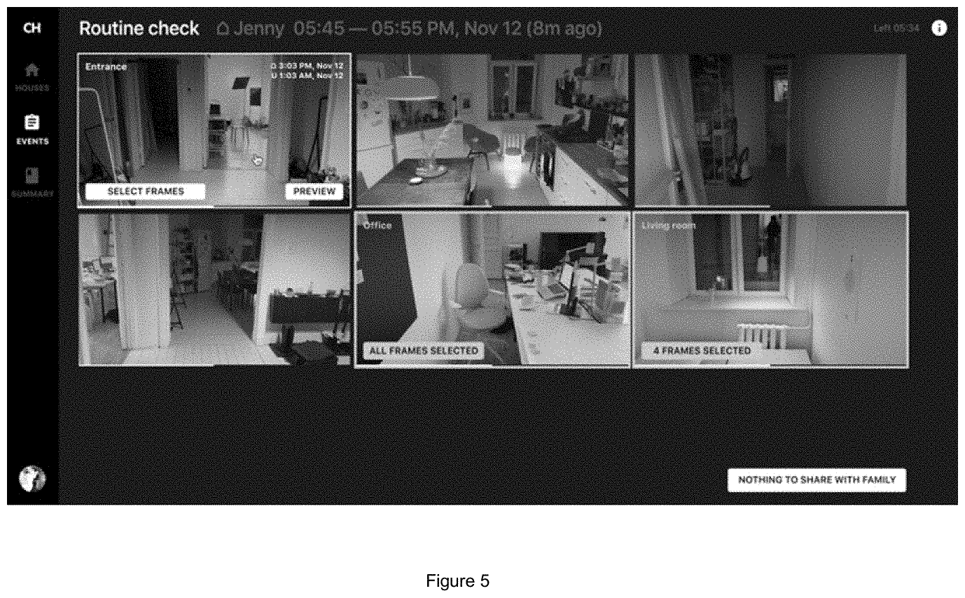

FIG. 5 depicts an application example of selecting a portion of the captured data to be transmitted for further analysis or for alerting an individual in accordance with some embodiments. The processing unit 120 may determine that an abnormal event has occurred. The operator may select a portion of the collected data (i.e. video/audio), e.g., frames from e.g., office, living room, and entrance, to be transmitted to another person, e.g., family member, caretaker, etc. In other words, the action of selecting a portion of the monitored data is the appropriate action for the particular abnormal event, as identified. The machine learning engine 140 also receives this information and trains and generates its machine learning model 142 to emulate the operator once the machine learning model 142 is applied to real data.

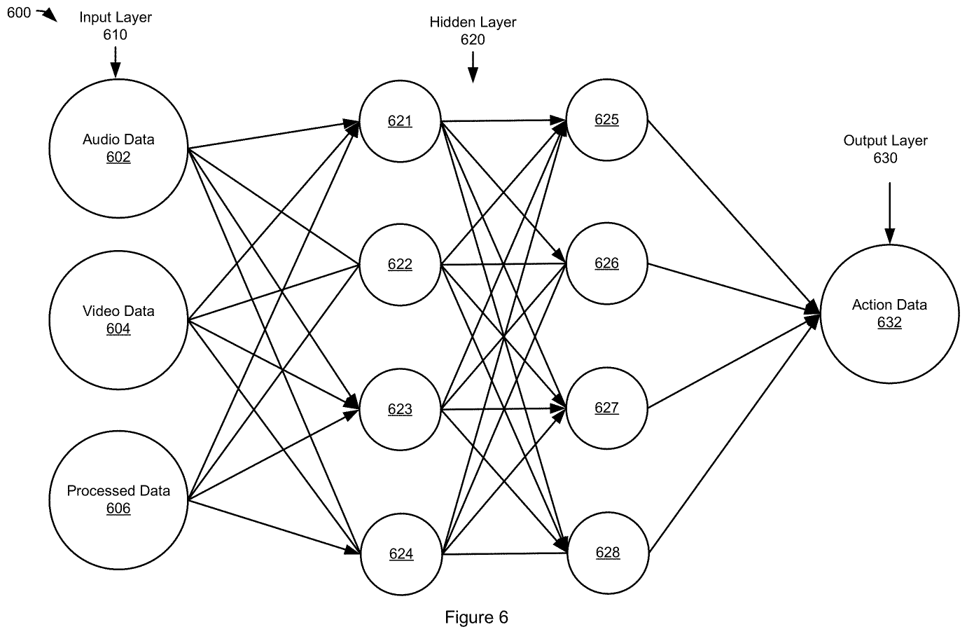

FIG. 6 depicts relational node diagram depicting an example of a neural network for identifying an abnormal event in accordance with some embodiments. In an example embodiment, the neural network 600 utilizes an input layer 610, one or more hidden layers 620, and an output layer 630 to train the machine learning model(s) or model to identify appropriate actions to be taken in response to the determined abnormal event from a captured input data, e.g., audio data, video data, infrared data, etc. In some embodiments, where the appropriate action to the abnormal event, as described above, have already been confirmed, supervised learning is used such that known input data, a weighted matrix, and known output data are used to gradually adjust the model to accurately compute the already known output. Once the model is trained, field data is applied as input to the model and a predicted output is generated. In other embodiments, where the appropriate action to the abnormal event has not yet been confirmed, unstructured learning is used such that a model attempts to reconstruct known input data over time in order to learn. Noted that FIG. 6 is described here as a structured learning model for depiction purposes and is not intended to be limiting.

In some embodiments, training of the neural network 600 using one or more training input matrices, a weight matrix, and one or more known outputs is initiated by one or more computers associated with the monitoring system. In an embodiment, a server may run known input data through a deep neural network in an attempt to compute a particular known output. For a non-limiting example, a server uses a first training input matrix and a default weight matrix to compute an output. If the output of the deep neural network does not match the corresponding known output of the first training input matrix, the server adjusts the weight matrix, such as by using stochastic gradient descent, to slowly adjust the weight matrix over time. The server computer then re-computes another output from the deep neural network with the input training matrix and the adjusted weight matrix. This process continues until the computer output matches the corresponding known output. The server computer then repeats this process for each training input dataset until a fully trained model is generated.

In the example of FIG. 6, the input layer 610 includes a plurality of training datasets that are stored as a plurality of training input matrices in a database associated with the monitoring system. The training input data includes, for example, audio data 602 from individuals being monitored, video data 604 from individuals being monitored, and processed data 606 as determined by the processing unit 120 to contain abnormal event within the monitored premises and so forth. Any type of input data can be used to train the model.

In some embodiments, audio data 602 is used as one type of input data to train the model, which is described above. In some embodiments, video data 604 are also used as another type of input data to train the model, as described above. Moreover, in some embodiments, processed data 606 are also used as another type of input data to train the model, as described above.

In some embodiments of FIG. 6, hidden layers 620 represent various computational nodes 621, 622, 623, 624, 625, 626, 627, 628. The lines between each node 621, 622, 623, 624, 625, 626, 627, 628 represent weighted relationships based on the weight matrix. As discussed above, the weight of each line is adjusted overtime as the model is trained. While the embodiment of FIG. 6 features two hidden layers 620, the number of hidden layers is not intended to be limiting. For example, one hidden layer, three hidden layers, ten hidden layers, or any other number of hidden layers may be used for a standard or deep neural network. The example of FIG. 6 also features an output layer 630 with the action data 632, which is the appropriate actions to be taken for abnormal events, as the known output. The action data 632 indicates the appropriate actions to be taken for the particular abnormal event for a given monitoring system. For example, the action data 632 may be a certain action, e.g., initiating a call, initiating a text, initiating a two way communication, alerting an individual, transmitting a portion of the data, etc., based on the audio data 602, video data 604, and/or processed data 606 as the input data. As discussed above, in this structured model, the action data 632 is used as a target output for continuously adjusting the weighted relationships of the model. When the model successfully outputs the action data 632, then the model has been trained and may be used to process live or field data.

Once the neural network 600 of FIG. 6 is trained, the trained model will accept field data at the input layer 610, such as audio data and video data and/or processed data from the monitoring system. In some embodiments, the field data is live data that is accumulated in real time. In other embodiments, the field data may be current data that has been saved in an associated database. The trained model is applied to the field data in order to generate one or more model for appropriate actions to be taken for one or more abnormal events at the output layer 630. Moreover, a trained model can determine that changing the model is appropriate as more data is processed and accumulated over time. Consequently, the trained model will determine the appropriate actions to be taken for a particular abnormal event over time and based on a specific monitored area and tailored to the premises being monitored. It is appreciated that the derived model may be stored in the machine learning model module within the processing unit 120 for execution by the respective processing unit once live data is being received.

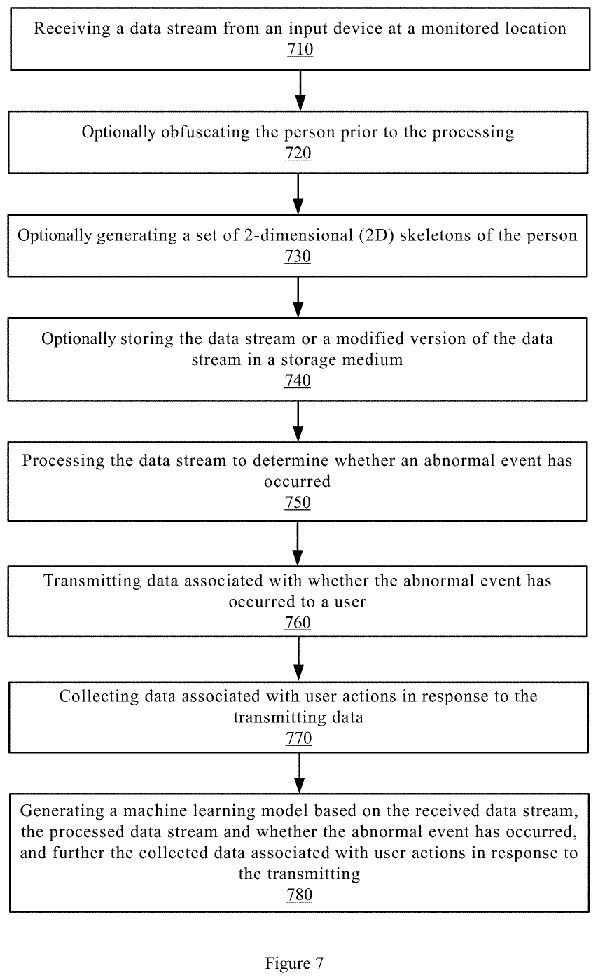

FIG. 7 depicts a flow chart illustrating an example of method flow for determining an abnormal event in accordance with some embodiments. At step 710, a data stream, e.g., video stream, audio stream, infrared data, etc., from an input device at a monitored location is received, as described above. At step 720, the received data is optionally obfuscated. For example, the individual being monitored is pixelated. At step 730, a 2-D skeletons of the person is optionally generated from the received data stream. As such, the privacy of the individuals being monitored are protected or the processing speed is increased. At step 740, the received data stream or the modified version thereof is optionally stored in a storage medium. At step 750, the data stream or modified version thereof is processed to determine whether an abnormal event has occurred. At step 760, data associated with whether the abnormal event has occurred is transmitted to a user. At step 770, data associated with the user (i.e. operator) actions, e.g., initiating a call, texting, transmitting a portion of the monitored frames, etc., in response to the transmitting of the data is collected. At step 780, a machine learning model is generated based on the received data stream, the processed data stream and whether the abnormal event has occurred, and further based on the collected data associated with the user actions in response to the transmitting. It is appreciated that in some embodiments the generated machine learning model is applied to subsequent processed data to determine appropriate actions to be performed. As described above, the machine learning model is further generated based on data stored in a database (i.e. data from other monitored locations and other users and/or controlled and supervised data).

It is appreciated that one embodiment may be implemented using a conventional general purpose or a specialized digital computer or microprocessor(s) programmed according to the teachings of the present disclosure, as will be apparent to those skilled in the computer art. Appropriate software coding can readily be prepared by skilled programmers based on the teachings of the present disclosure, as will be apparent to those skilled in the software art. The invention may also be implemented by the preparation of integrated circuits or by interconnecting an appropriate network of conventional component circuits, as will be readily apparent to those skilled in the art.

The methods and system described herein may be at least partially embodied in the form of computer-implemented processes and apparatus for practicing those processes. The disclosed methods may also be at least partially embodied in the form of tangible, non-transitory machine readable storage media encoded with computer program code. The media may include, for example, RAMs, ROMs, CD-ROMs, DVD-ROMs, BD-ROMs, hard disk drives, flash memories, or any other non-transitory machine-readable storage medium, wherein, when the computer program code is loaded into and executed by a computer, the computer becomes an apparatus for practicing the method. The methods may also be at least partially embodied in the form of a computer into which computer program code is loaded and/or executed, such that, the computer becomes a special purpose computer for practicing the methods. When implemented on a general-purpose processor, the computer program code segments configure the processor to create specific logic circuits. The methods may alternatively be at least partially embodied in a digital signal processor formed of application specific integrated circuits for performing the methods.

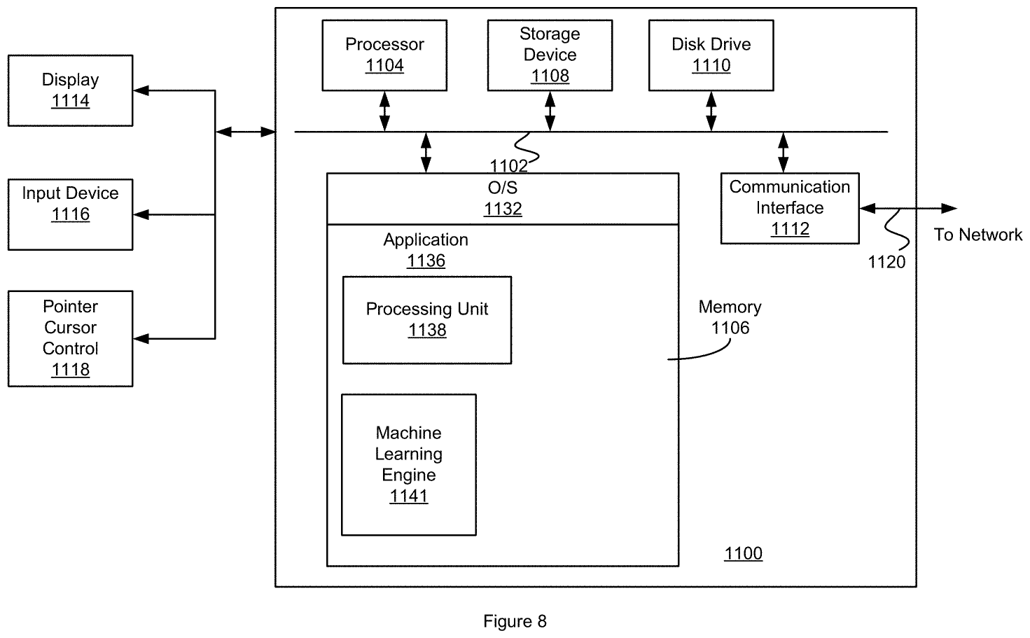

FIG. 8 depicts a block diagram depicting an example of computer system suitable for generating a machine learning model and determining appropriate actions to an abnormal event in accordance with some embodiments. In some examples, computer system 1100 can be used to implement computer programs, applications, methods, processes, or other software to perform the above-described techniques and to realize the structures described herein. Computer system 1100 includes a bus 1102 or other communication mechanism for communicating information, which interconnects subsystems and devices, such as a processor 1104, a system memory (“memory”) 1106, a storage device 1108 (e.g., ROM), a disk drive 1110 (e.g., magnetic or optical), a communication interface 1112 (e.g., modem or Ethernet card), a display 1114 (e.g., CRT or LCD), an input device 1116 (e.g., keyboard), and a pointer cursor control 1118 (e.g., mouse or trackball). In one embodiment, pointer cursor control 1118 invokes one or more commands that, at least in part, modify the rules stored, for example in memory 1106, to define the electronic message preview process.

According to some examples, computer system 1100 performs specific operations in which processor 1104 executes one or more sequences of one or more instructions stored in system memory 1106. Such instructions can be read into system memory 1106 from another computer readable medium, such as static storage device 1108 or disk drive 1110. In some examples, hard-wired circuitry can be used in place of or in combination with software instructions for implementation. In the example shown, system memory 1106 includes modules of executable instructions for implementing an operating system (“OS”) 1132, an application 1136 (e.g., a host, server, web services-based, distributed (i.e., enterprise) application programming interface (“API”), program, procedure or others). Further, application 1136 includes a module of executable instructions for a processing unit 1138 that determines whether an abnormal event has occurred and a machine learning engine 1141 to train and generate a machine learning model based on the monitored data, the determined abnormal event(s), and actions taken by an operator.

The term “computer readable medium” refers, at least in one embodiment, to any medium that participates in providing instructions to processor 1104 for execution. Such a medium can take many forms, including but not limited to, non-volatile media, volatile media, and transmission media. Non-volatile media includes, for example, optical or magnetic disks, such as disk drive 1110. Volatile media includes dynamic memory, such as system memory 1106. Transmission media includes coaxial cables, copper wire, and fiber optics, including wires that comprise bus 1102. Transmission media can also take the form of acoustic or light waves, such as those generated during radio wave and infrared data communications.

Common forms of computer readable media include, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, electromagnetic waveforms, or any other medium from which a computer can read.

In some examples, execution of the sequences of instructions can be performed by a single computer system 1100. According to some examples, two or more computer systems 1100 coupled by communication link 1120 (e.g., LAN, PSTN, or wireless network) can perform the sequence of instructions in coordination with one another. Computer system 1100 can transmit and receive messages, data, and instructions, including program code (i.e., application code) through communication link 1120 and communication interface 1112. Received program code can be executed by processor 1104 as it is received, and/or stored in disk drive 1110, or other non-volatile storage for later execution. In one embodiment, system 1100 is implemented as a hand-held device. But in other embodiments, system 1100 can be implemented as a personal computer (i.e., a desktop computer) or any other computing device. In at least one embodiment, any of the above-described delivery systems can be implemented as a single system 1100 or can implemented in a distributed architecture including multiple systems 1100.

In other examples, the systems, as described above can be implemented from a personal computer, a computing device, a mobile device, a mobile telephone, a facsimile device, a personal digital assistant (“PDA”) or other electronic device.

In at least some of the embodiments, the structures and/or functions of any of the above-described interfaces and panels can be implemented in software, hardware, firmware, circuitry, or a combination thereof. Note that the structures and constituent elements shown throughout, as well as their functionality, can be aggregated with one or more other structures or elements.

Alternatively, the elements and their functionality can be subdivided into constituent sub-elements, if any. As software, the above-described techniques can be implemented using various types of programming or formatting languages, frameworks, syntax, applications, protocols, objects, or techniques, including C, Objective C, C++, C #, Flex™, Fireworks®, Java™, Javascript™, AJAX, COBOL, Fortran, ADA, XML, HTML, DHTML, XHTML, HTTP, XMPP, and others. These can be varied and are not limited to the examples or descriptions provided.

While the embodiments have been described and/or illustrated by means of particular examples, and while these embodiments and/or examples have been described in considerable detail, it is not the intention of the Applicants to restrict or in any way limit the scope of the embodiments to such detail. Additional adaptations and/or modifications of the embodiments may readily appear to persons having ordinary skill in the art to which the embodiments pertain, and, in its broader aspects, the embodiments may encompass these adaptations and/or modifications. Accordingly, departures may be made from the foregoing embodiments and/or examples without departing from the scope of the concepts described herein. The implementations described above and other implementations are within the scope of the following claims.

What is claimed is:

1. A method comprising:
receiving a data stream from an input device at a monitored location;
processing the data stream to determine whether an abnormal event has occurred;
transmitting data associated with whether the abnormal event has occurred to a user;
collecting data associated with user actions in response to the transmitting data; and
generating a machine learning model based on the received data stream, the processed data stream and whether the abnormal event has occurred, and further the collected data associated with user actions in response to the transmitting.

2. The method of claim 1, wherein the data stream includes a video stream and audio stream.

3. The method of claim 1 further comprising obfuscating a portion of the data stream prior to the processing.

4. The method of claim 3, wherein the obfuscation includes generating a set of 2-dimensional (2D) skeletons of the person or pixelating an individual in the data stream.

5. The method of claim 1, wherein the input device includes a camera and a microphone.

6. The method of claim 1 further comprising applying the machine learning model to subsequent processed data that determine whether a subsequent abnormal event has occurred to determine appropriate actions to be performed.

7. The method of claim 6, wherein the appropriate actions include automatically communicating with an individual within the data stream at the monitored location, automatically calling an emergency service, or automatically transmitting a message to another user.

8. The method of claim 1, wherein the machine learning model includes clustering and grouping model.

9. The method of claim 1 further comprising receiving a plurality of other actions from a database, wherein the plurality of other actions includes appropriate actions in response to a plurality of abnormal events, and wherein the generating the machine learning model is further based on the plurality of other actions.

10. The method of claim 1 further comprising storing the generated machine learning model.

11. A method comprising:
receiving a data stream associated with a monitored location;
processing the data stream to determine whether an abnormal event has occurred;
transmitting data associated with whether the abnormal event has occurred to a user;
collecting data associated with user actions in response to the transmitting data; and
generating a machine learning model based on the received data stream, the processed data stream and whether the abnormal event has occurred, and further the collected data associated with user actions in response to the transmitting.

12. The method of claim 11, wherein the data stream includes a video stream and audio stream.

13. The method of claim 11 further comprising obfuscating a portion of the data stream prior to the processing.

14. The method of claim 13, wherein the obfuscation includes generating a set of 2-dimensional (2D) skeletons of the person or pixelating an individual in the data stream.

15. The method of claim 11 further comprising applying the machine learning model to subsequent processed data that determine whether a subsequent abnormal event has occurred to determine appropriate actions to be performed.

16. The method of claim 15, wherein the appropriate actions include automatically communicating with an individual within the data stream at the monitored location, automatically calling an emergency service, or automatically transmitting a message to another user.

17. The method of claim 11, wherein the machine learning model includes clustering and grouping model.

18. The method of claim 11 further comprising receiving a plurality of other actions from a database, wherein the plurality of other actions includes appropriate actions in response to a plurality of abnormal events, and wherein the generating the machine learning model is further based on the plurality of other actions.

19. The method of claim 11 further comprising storing the generated machine learning model.

20. A system comprising:
a data capturing system configured to capture a video/audio data at a monitored location;
a processing unit configured to receive the video/audio data and determine whether an abnormal event has occurred, and wherein the processing unit is further configured to transmit a signal to a user based on a determination whether the abnormal event has occurred; and
a machine learning engine configured to receive actions taken by the user, wherein the machine learning engine is further configured to receive the video/audio data and data associated with the determination whether the abnormal event has occurred, and wherein the machine learning engine is further configured to generate a machine learning model based on the received data.

21. The system of claim 20 further comprising an obfuscation engine configured to obfuscate a portion of the video/audio data.

22. The system of claim 20, wherein the data capturing system includes a camera and a microphone.

23. The system of claim 20, wherein the machine learning engine is further configured to apply the machine learning model to subsequent processed data from the processing unit to determine appropriate actions to be performed.

24. The system of claim 23, wherein the appropriate actions include automatically communicating with an individual within the data stream at the monitored location, automatically calling an emergency service, or automatically transmitting a message to another user.

25. The system of claim 20, wherein the machine learning model includes clustering and grouping model.

26. The system of claim 20 wherein the machine learning engine is further configured to receive a plurality of other actions from a database, wherein the plurality of other actions includes appropriate actions in response to a plurality of abnormal events, and wherein the machine learning model is further generated based on the plurality of other actions.

27. The system of claim 20, wherein the machine learning engine is further configured to store the generated machine learning model.
